(19) United States
(12) Patent Application Publication
(10) Pub. No.: US 2023/0158206 A1
(43) Pub. Date: May 25, 2023

(54) DECELLULARIZED MENISCAL CARTILAGE AND USES THEROF

(71) Applicant: The Children's Hospital of Philadelphia, Philadelphia, PA (US)

(72) Inventors: Riccardo L. GOTTARDI, Philadelphia, PA (US); Paul M. GEHRET, Philadelphia, PA (US)

(73) Assignee: The Children's Hospital of Philadelphia, Philadelphia, PA (US)

(21) Appl. No.: 17/916,998

(22) PCT Filed: Apr. 5, 2021
(86) PCT No.: PCT/US21/25782

§ 371 Date: Oct. 4, 2022

Related U.S. Application Data
(60) Provisional application No. 63/005,762, filed on Apr. 6, 2020.

(51) Int. Cl. A61L 27/36; A61L 27/38

(52) U.S. Cl. CPC A61L 27/3612; A61L 27/3687; A61L 27/3804; A61L 2300/412; A61L 2430/40

(57) ABSTRACT

The present disclosure provides improved biomaterials extracted from fibrous meniscal cartilage (FMC). The materials are at least partially decellularized and enzyme treated to remove at least a portion of the elastin and blood vessels found in FMC. Such biomaterials can be employed as tissue scaffolds, such as in transplant procedures.

### PRIORITY CLAIM

This application claims benefit of priority to U.S. Provisional Application Ser. No. 63/005,762, filed Apr. 6, 2020, the entire content of which is hereby incorporated by reference in its entirely.

### BACKGROUND

### 1. Field

The present disclosure relates generally to the field of cell and tissue biology, medicine, and medical procedures such as transplants. More particularly, it concerns improved biomaterials extracted from meniscal cartilage that have improved properties and their use in the transplant surgery.

### 2. Description of Related Art

Tissue engineering uses scaffolds composed of natural and polymeric materials that are seeded with cells to generate new tissues for organ replacement. In cartilage tissue engineering, most of these approaches have all fallen short in recapitulating the native extracellular matrix, failing to provide sufficient mechanical strength and inductive signals to promote cell lineage commitment. Recently, scientists have been using decellularized tissues as a scaffold to recapitulate the native environment. This technique has shown promising results and has even began to translate to the clinic in the form of heart valves and skin. However, hyaline cartilage tissue, whose dry weight consisting mostly of collagen II and glycosaminoglycans (GAGs) is far too dense to for cells to reinvade once decellularized.

The generation of natural channels inside cartilage has been shown (Lehmann et al., 2019; Nurberger et al., 2019). In this method, elastin fibers are removed from elastic cartilage found in the ear or nose. Cells then reinvade the elastic cartilage via the channels from the elastase. This approach uses elastic cartilage, is limited to the removal of elastin, relies on the use of bovine auricular cartilage (xenogeneic) as scaffold material. Improved compositions and methods for the creation of acellular cartilage tissue grafts are thus needed.

### SUMMARY

In one aspect, the present disclosure provides a method of preparing a decellularized transplant material comprising (a) providing fibrous meniscal cartilage (FMC) or intervertebral annulus fibrosis cartilage (IAFC); and (b) treating said FMC or IAFC with to remove blood vessels or elastin or both blood vessels and elastin to produce said decellularized transplant material. Step (b) may comprise treatment with one or more enzymes such an endopeptidase (e.g., trypsin, chymotrypsin), a cysteine protein (e.g., papain) and in particular with pepsin and/or elastase. Step (b) may instead comprise treatment with EDTA, EGTA, DCTA, an acid (e.g., acetic acid, hydrochloric acid) or base (e.g., NaOH).

The FMC of step (a) may be of human origin or may be of non-human origin, such as pig, rabbit, sheep, goat and cow. The FMC or IAFC of step (a) may be cadaver FMC or IAFC or from a living donor. The decellularized transplant material may be free of at least 50%, 60%, 70%, 75%, 80%, 85%, 90%, 95% or 99% greater of starting elastin content or may be 100% devoid of starting elastin content, and/or is free of at least 50%, 60%, 70%, 75%, 80%, 85%, 90%, 95% or 99% greater of starting blood vessel content or may be 100% devoid of starting blood vessel content by dry weight. The decellularized transplant material may be free of at least 50%, 60%, 70%, 75%, 80%, 85%, 90%, 95% or 99% greater of starting cellular content by dry weight. The decellularized transplant material maybe at least 30%, 40%, 50%, 60%, 70%, 75%, 80%, 85%, 90%, 95%, 99% or 100% glycosaminoglycan and collagen by dry weight.

The FMC or IAFC of step (a) may be treated by one or more freeze/thaw cycles. Step (b) may comprise treatment with pepsin followed by treatment with elastin, such as wherein treatment with pepsin is for about 24 hours at 37° C. and/or treatment with elastin is for about 24 hours at 37° C. The method may further comprise incubating the decellularized transplant material in serum, such as FBS, or with any useful peptide, protein, small molecule, growth factor, or chemically modifying agent. The method may further comprise storage about +4 to −80 ° C., such as at −20 ° C. The method may further comprise the step of reintroducing cells into said decellularized transplant material following step (b).

In another embodiment, there is provided a decellularized transplant material made according to the method as defined herein.

In yet another embodiment, there is provided a decellularized transplant material comprising fibrous meniscal cartilage (FMC) or intervertebral annulus fibrosis cartilage (IAFC) that lacks at least 50% of the elastin and blood vessels of normal FMC or IAFC. The FMC or IAFC of step (a) may be of human origin or may be of non-human origin, such as pig, rabbit, sheep, goat and cow. The FMC or IAFC of step (a) may be cadaver FMC or IAFC or from a living donor. The decellularized transplant material may be free of at least 50%, 60%, 70%, 75%, 80%, 85%, 90%, 95% or 99% greater of starting elastin content or may be 100% devoid of starting elastin content, and/or may be free of at least 50%, 60%, 70%, 75%, 80%, 85%, 90%, 95% or 99% greater of starting blood vessel content or may be 100% devoid of starting blood vessel content by dry weight. The decellularized transplant material may be free of at least 50%, 60%, 70%, 75%, 80%, 85%, 90%, 95% or 99% greater of starting cellular content by dry weight. The decellularized transplant material may be at least 30%, 40%, 50%, 60%, 70%, 75%, 80%, 85%, 90%, 95%, 99% or 100% glycosaminoglycan and collagen by dry weight. The decellularized transplant material may be re-cellularized with cell, such as a stem cell (such as mesenchymal stem cells), a progenitor cell, chondrocytes, fibrochondrocytes, cartilage progenitor cells, induced pluripotent stem cells, stem/progenitor cells derived from induced pluripotent stem cells, synovial stem cells, pericytes, pulp/gingival stem cells, adipose derived stem cells, fibroblasts, endothelial cells, muscle cells, osteoblasts, osteocytes, osteoclasts, macrophages, monocytes, or cells of the immune system. The material may be frozen.

In yet another embodiment, there is provided a method of transplanting a decellularized transplant material into a living subject comprising (a) obtaining a decellularized transplant material as defined herein; and (b) transplanting said material into said subject. The method may further comprise the step of reintroducing cells into said decellularized transplant material prior to step (b). The re-cellularized transplant material may be transplanted immediately after reintroducing cells and without culture. The method may further comprise culturing the re-cellularized transplant material prior to step (b), optionally including the use of one or more factors or conditions that induce cell differentiation/specification.

Culturing may be for 1 day to about 3 months, such as about 6 weeks. The decellularized transplant material may be re-cellularized with a stem cell (such as mesenchymal stem cells), a progenitor cell, chondrocytes, fibrochondrocytes, cartilage progenitor cells, induced pluripotent stem cells, stem/progenitor cells derived from induced pluripotent stem cells, synovial stem cells, pericytes, pulp/gingival stem cells, adipose derived stem cells. The re-introduced cells may be autologous to said subject, such as genetically engineered or modified, such as iPSCs, or the re-introduced cells may be allogenic to said subject. About 1×10³ to about 1×10⁸ cells may be reintroduced.

The decellularized transplant material may be autologous, allogenic or xenogenic to said subject. The subject may be a non-human animal or a human, such as a human pediatric subject. The decellularized transplant material may be transplanted into trachea, larynx, rib, ear (e.g., tympanic membrane), nose, hip, knee, temporomandibular joint, epiglottis, intervertebral disc, a joint, or meniscus. The decellularized transplant material may be transplanted into a bone defect. The subject may have suffered a traumatic injury or undergone resection of a cancerous lesion. The decellularized transplant material may be transplanted as a treatment for disk herniation, tympanic membrane damage, laryngoesophageal fistula, cleft palate, osteoarthritis, spine fusion, joint overuse, a birth defect (e.g., CHARGE syndrome), or microtia, or as an alveolar bone graft.

In particular, the subject may have a condition selected from disk herniation, conductive hearing loss cause by tympanic membrane defects, laryngoesophageal fistula, or tracheostomy.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description. Note that simply because a particular compound is ascribed to one particular generic formula doesn't mean that it cannot also belong to another generic formula.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-D. (FIG. 1A) The meniscus is a vascularized piece of fibrocartilage. Within this structure there are both elastin fibers and blood vessels. These components will be removed using enzymes and/or chemicals to create channels for re-cellularization. Cells will reinvade the channel laden meniscal cartilage and mature to form new cartilage. The mature, functional cartilage will then be used in the clinic. (FIG. 1B) Isolation of the meniscus from a pig's knee. (FIG. 1C) Close up images of the medial and lateral meniscus. (FIG. 1D) Cross section of the meniscus. The blood vessels can be clearly seen invading the posterior end of the tissue.

FIGS. 2A-C. (FIG. 2A) Verhoeff van gieson stain of a cross-section of the center region of the meniscus. Purple and black coloring indicates elastin and the pink indicates collagen. (FIG. 2B) Verhoeff van gieson stain of a circumferential section of the center region of the meniscus. (FIG. 2C) Quantification of the amount of blood vessels and elastin present in each region of the meniscus. Percent abundance was calculated through the ratio of the area of the elastin fibers and blood vessel to the total area. Significant differences were calculated using a Tukey's Honest Significant Difference Test (***=p<0.001, **p<0.01, *=p<0.05. (FIG. 2A, FIG. 2B & FIG. 2C) Scale bar=1 mm.

FIGS. 3A-E. (FIG. 3A & FIG. 3B) Cross-sectional view of the meniscus stained with the Verhoeff-Van Gieson before and after pepsin and elastase treatment, respectively. The pink regions indicate collagen and purple regions indicate elastin. (FIG. 3C & FIG. 3D) Circumferential view of the meniscus stained with the Verhoeff-Van Gieson before and after pepsin and elastase treatment, respectively. The pink regions indicate collagen and purple regions indicate elastin. The elastin and blood vessels removed with the enzymatic treatment was measured via image analysis. (FIG. 3E) The free space area is measured in imagej and compared to untreated tissue and decellularized tissue. Significant differences were calculated using a Tukey's Honest Significant Difference Test. (***=p<0.001, **p<0.01, *=p<0.05). (FIG. 3A & FIG. 3B) Scale bar=1 mm (FIG. 3C & FIG. 3D) Scale bar=200 μm.

FIGS. 4A-G. (FIG. 4A) Cylinders of decellularized, channel-laden meniscus were punched from a digested and treated meniscus cross-section. (FIG. 4B) A transwell plate with a gradient ranging from 0-20% serum is used to cause cells to invade into the channels. (FIG. 4C) Meniscus cylinders inside the transwell plate after cells are seeded on top. (FIG. 4D) Calcein AM live cell stain of cells on the meniscus cylinder one week after seeding. (FIG. 4E) Calcein AM live cell stain of cells in the meniscus cross-section one week after seeding. (FIG. 4F) Calcein AM live cell stain of cells on the meniscus cylinder two weeks after seeding. (FIG. 4G) Calcein AM live cell stain of cells in the meniscus cross section 2 weeks week after seeding.

FIGS. 5A-D. H & E stained cross-sections of cells invading the decellularize channel laden meniscus after 1 week. Scale bar=200 μm.

FIG. 6. Overview of the current proposed technology. The meniscus is a vascularized piece of fibrocartilage. Within this structure there are both elastin fibers and blood vessels. (1) These components will be removed using enzymes to create channels for re-cellularization. (2) Cells will reinvade the channel laden meniscal cartilage and mature to form new cartilage. (3) The mature, functional cartilage will then be used in the clinic.

FIGS. 7A-D. (FIGS. 7A-B) Isolation of the meniscus from a pig's knee obtained from a local butcher. (FIG. 7C) Close up images of the medial and lateral meniscus. Cross section of the meniscus. The blood vessels can be seen invading the posterior end of the tissue. (FIG. 7D) Those same blood vessels can be seen in black and purple. The radial meniscus cross-section was stained with Verhoef van gieson elastic fiber stain.

FIGS. 8A-B. (FIGS. 8A-B) Radial meniscus cross-section following the removal of the enzymatic digestion of elastic fibers. Large channels are observed where the blood vessels were previously as well as smaller channels throughout the tissue. These channels will be utilized for cellular re-invasion into the decellularized mensicus

FIGS. 9A-F. (FIGS. 9A-B) Verhoef van gieson stained radial meniscus cross sections (FIG. 9A) before and (FIG. 9B) after treatment with pepsin and elastase. These images show the removal of elastic fibers as well as the creation of channels for re-cellularization. (FIGS. 9C-D) Verhoef van gieson stained circumferential meniscus cross sections (FIG. 9C) before and (FIG. 9D) after treatment with pepsin and elastase. (FIGS. 9E-F) H&E stained circumferential meniscus cross sections (FIG. 9E) before and (FIG. 9F) after treatment with pepsin and elastase. This orientation represents the direction cells will be seeded in. The channels are well aligned and well sized.

FIGS. 10A-C. (FIG. 10A) The total amount of empty area within the decellularized, digested meniscus (MenD) was measured and compared to the total area of the section to calculate percent area that is occupied by the channels. MenD had significantly more “empty space” than the native meniscus. (FIG. 10B) Same comparison as FIG. 10A but looking at porosity. (FIG. 10C) The percent channel area from FIG. 10A was verified using water displacement method to calculate the porosity of MenD. The water displacement showed a significant increase in MenD porosity.

FIGS. 11A-F. (FIGS. 11A-B) Verhoef van gieson stained radial meniscus cross sections before and after digestion to exemplify the loss of elastin within the meniscus when enzymatically degraded. (FIG. 11C) Quantification of this loss of elastin within the meniscus post digestion using a commercially available kit. (FIGS. 11D-E) DAPI stained radial meniscus cross sections before and after digestion to exemplify the loss of DNA following the decellularization process. (FIG. 11F) Quantification of this loss of DNA in MenD. The total DNA is below 50 ng/mg of tissue which is the known threshold to prevent immune responses in vivo.

FIGS. 12A-F. (FIGS. 12A-B) Alcian stained radial meniscus cross sections before and after digestion to exemplify the loss of glycosaminoglycans (GAGs) within the meniscus when enzymatically degraded. (FIG. 12C) Quantification of this loss of GAGs post digestion using a DMMB assay. (FIG. 12D-E) Sirus stained radial meniscus cross sections before and after digestion to exemplify the stability of Collagen following the decellularization process. (FIG. 12F) Quantification of the collagen content post digestion using a hydroxyproline assay.

FIGS. 13A-B. The (FIG. 13A) bulk and (FIG. 13B) dynamic compressive modulus of the native meniscus versus MenD. Both the radial and circumferential directions were tested due to the anisotropy of the meniscus. MenD exhibited a significant decrease in bulk mechanical strength due to the formation of channels; however, it maintained its dynamic modulus as shown by being statistically insignificant form native meniscus.

FIGS. 14A-C. MenD reinvasion with cells. (FIG. 14A) Scaffolds were punched from the digested, decellularized meniscus using a biopsy punch. (FIG. 14B) The MenD scaffolds were added to a transwell plate for cell seeded. (FIG. 14C) Cells were placed on top of the scaffold in media fetal bovine serum (FBS). To create a gradient for invasion, 20% media with serum was added before the transwell insert to act as a chemoattractant for the cells seeded on top of the scaffold.

FIG. 15. Live cell staining (calcein AM) of cells seeded on top of MenD at varying cell densities (100,000-600,000 cells per 6 mm scaffold). The overview, surface and cross-section of imaged to examine cellular interaction with MenD. All the cells were alive and can be see invading MenD.

FIG. 16. Cell post invasion within MenD constructs based on seeding density. Scaffolds with 200,000-400,000 had the most cell invasion.

FIGS. 17A-C. (FIGS. 17A-C). Cell invasion 1 Day (FIG. 17A), 3 Days (FIG. 17B), and 5 Days (FIG. 17C) of hMSC invasion. Images to the left are a DAPI (Blue) and collagen autofluorescence (Red) overlay. Images to the right are DAPI (blue) only images. After 3 Days the cell densities within MenD achieve a homogenous cell distribution with similar densities. This indicates that 3 days of invasion is necessary full MenD repopulation.

FIGS. 18A-B. (FIG. 18A) Hematoxylin and eosin (H&E) staining of a scaffold cross-section following 3 Days of MenD invasion. (FIG. 18B) H & E staining of the native meniscus. These images show that MenD achieves a similar cell density to the native meniscus following 3 days of invasion.

FIGS. 19A-E. (FIG. 19A) The hMSC density of cells seeded in MenD for 1, 3, 5, or 7 days. This graph indicates that MenD constructs achieve a maximum cell density at 3 days of invasion. (FIGS. 19B-E) The distribution of cells throughout the scaffold at 1, 3 or 5 days (FIGS. 19B-D) or in the native meniscus. These graphs indicate that the cells achieve a homogenous distribution starting at day 3 and resembles native meniscus tissue.

FIGS. 20A-C. Live dead (calcein AM) of cartilage progenitor cells invaded into MenD for 3 days then differentiated using chondrogenic media for 3 weeks. The overview (FIG. 20A-top left), surface (FIG. 20B), below the surface (FIG. 20C), and cross-section (FIG. 20A-bottom) are shown. These images indicate that the cells have stayed alive within MenD and began to create engineered cartilage from the MenD scaffold.

FIGS. 21A-D. MenD utilized as a graft material for a laryngotracheal reconstruction. During this operation rabbit upper airways were exposed to show the larynx, cricoid, and 1st tracheal ring (FIG. 21A). An acellular (FIG. 21B), 3 day re-cellularized (FIG. 21C), and 3 week differentiated (FIG. 21D) MenD scaffold was sutured into the cricoid and first tracheal ring. The increase in stiffness of the MenD constructs as cells are added and differentiation is shown based on the amount of construct can expand the airway during this operation.

FIGS. 22A-F. Endoscope images of Day 3 acellular MenD in the airway (FIG. 22A), Day 82 acellular MenD (FIG. 22B), and Day 82 cellular MenD in the airway. Overview images of the explanted airways after 82 days with acellular (FIG. 22D) or cellular (FIG. 22E) MenD. Native explanted airway for comparison (FIG. 22F). As seen from the endoscope images the MenD construct epithelializes after 82 days and expands the airway substantially. Upon ex vivo examination, these grafts have become more cartilaginous and are beginning to resemble a native airway again.

FIGS. 23A-F. H&E stained airway cross-sections of (FIG. 23A & FIG. 23D) Day 3 acellular MenD, (FIG. 23B & FIG. 23E) Day 82 acellular MenD, and (FIG. 23C & FIG. 23F) native airway cartilage. The red dotted line indicates the boundary between the native tissue and the scaffold. B&C show significant airway regeneration within the scaffold after 3 months as marked by the yellow asterisk. This regeneration protrudes for into the scaffold and indicates that the airway is remodeling the MenD scaffold into new hyaline airway cartilage.

FIGS. 24A-F. Saf-O stained airway cross-sections of (FIG. 24A & FIG. 24D) Day 3 acellular MenD, (FIG. 24B & FIG. 24E) Day 82 acellular MenD, and native airway cartilage (FIG. 24C & FIG. 24F). The red dotted lines indicate the boundary between the native tissue and the scaffold. FIG. 24B & FIG. 24E show significant airway regeneration within the scaffold after 3 months as marked by the yellow asterisk. This stain indicates that the regenerated cartilage in FIG. 24B & FIG. 24E contains substantial glycosaminoglycans further indicating that MenD regenerates hyaline cartilage.

### DESCRIPTION OF ILLUSTRATIVE EMBODIMENTS

As discussed above, tissue engineering is limited by the availability of materials that faithfully recapitulate the native extracellular matrix and thus fail to provide sufficient mechanical strength and inductive signals to promote cell lineage commitment. While the use of decellularized tissues as scaffolds has shown promising results, the use of hyaline cartilage tissue has also its shortcomings.

Here, the inventors have employed fibrous meniscal cartilage, a material that contains blood vessels and elastin fibers in addition to collagen and glycosaminoglycans (GAGs) extracellular matrix. To create channels within the meniscal cartilage, thereby opening the way for cellular invasion before or following transplant, blood vessels and elastin were removed by enzymatic degradation. After treatment, only collagen fibers and GAGs remain, and the resulting channels allow for cellular invasion. The technology is readily applicable to the engineering of functional ear, nose, hip, knee and other types of cartilage. In a particular aspect, this technology can be employed to create patient-specific cartilage for laryngeal tracheal reconstruction surgery (LTR), a surgery needed by pediatric patients who have severe narrowing of the trachea. Use of the engineered cartilage described herein would permit surgeries that expand the trachea, allowing for patients to breathe more easily.

In addition, the inventors have observed that the annulus fibrosis of the invertebrate disk is nearly identical to the meniscus in its biochemical composition. The process utilized to decellularize and create channels within the meniscal cartilage can also be employed to do the same with annulus fibrosis cartilage, which will provide a new potential geometry and fiber orientation option.

As mentioned above, one group has removed elastin fibers from elastic cartilage found in the ear or nose that permit some cellular invasion into channels formed by elastase treatment. That approach differs substantially from the one proposed here. For example, the present disclosure describes the use of fibrous cartilage, which is anatomically, structurally, and compositionally different than elastic cartilage. Also, this approach removes both blood vessels and elastin, which was known to be present but not previously known to be significantly abundant in fibrous cartilage, and blood vessels. Further, from a translational standpoint, the present approach lends itself well to the use of human meniscus (allogeneic) from biobanks and cadavers, among many possible sources, which can provide a significant volume of cartilage. All of these features make the methods described here a significant advance in the field. These and other features of the disclosure are described in detail below.

### I. Cartilage

Cartilage is a resilient and smooth elastic tissue, a rubber-like padding that covers and protects the ends of long bones at the joints, and is a structural component of the rib cage, the ear, the nose, the bronchial tubes, the intervertebral discs, and many other body components. It is not as hard and rigid as bone, but it is much stiffer and much less flexible than muscle. The matrix of cartilage is made up of glycosaminoglycans, proteoglycans, collagen fibers and, sometimes, elastin. Because of its rigidity, cartilage often serves the purpose of holding tubes open in the body. Examples include the rings of the trachea, such as the cricoid cartilage and carina.

Cartilage is composed of specialized cells called chondrocytes that produce a large amount of collagenous extracellular matrix, abundant ground substance that is rich in proteoglycan and elastin fibers. Cartilage is classified in three types—elastic cartilage, hyaline cartilage and fibrocartilage—which each differ in relative amounts of collagen and proteoglycan. Cartilage generally does not contain blood vessels (avascular) or nerves (aneural). Nutrition is supplied to the chondrocytes by diffusion. The compression of the articular cartilage or flexion of the elastic cartilage generates fluid flow, which assists diffusion of nutrients to the chondrocytes. Compared to other connective tissues, cartilage has a very slow turnover of its extracellular matrix and does not repair.

Meniscal cartilage is a specific type of elastic cartilage. The meniscus itself is a crescent-shaped fibrocartilaginous anatomical structure that, in contrast to an articular disc, only partly divides a joint cavity. In humans they are present in the knee, wrist, acromioclavicular, sternoclavicular, and temporomandibular joints; in other animals they may be present in other joints. Generally, the term “meniscus” is used to refer to the fibrocartilage in the knee, either to the lateral or medial meniscus. Both are cartilaginous tissues that provide structural integrity to the knee when it undergoes tension and torsion. The menisci are also known as “semi-lunar” cartilages, referring to their half-moon, crescent shape.

As an example, the menisci of the knee are two pads of fibrocartilaginous tissue which serve to disperse friction in the knee joint between the lower leg (tibia) and the thigh (femur). They are concave on the top and flat on the bottom, articulating with the tibia. They are attached to the small depressions (fossae) between the condyles of the tibia (intercondyloid fossa), and towards the center they are unattached and their shape narrows to a thin shelf. The blood flow of the meniscus is from the periphery (outside) to the central meniscus. Blood flow decreases with age and the central meniscus is avascular by adulthood, leading to very poor healing rates.

Another source material is the cartilage from intervertebral anulus fibrosus. This material consists of several layers (laminae) of fibrocartilage made up of both type I and type II collagen. Type I is concentrated toward the edge of the ring, where it provides greater strength. The stiff laminae can withstand compressive forces.

### II. Methods of Preparing Cartilage Transplant Materials

The transplant materials described herein can be prepared from any suitable source. For example, the materials may be obtained from living or deceased donors and can be obtained from human or non-human (e.g., animal) tissues. The components can be also obtained from commercial sources. The components can be purified, substantially purified, partially purified, or non-purified. Various treatment steps are discussed below.

### A. Freezing and Lyophilization

The fibrous meniscal cartilage (FMC) intervertebral annulus fibrosis cartilage (IAFC) can be washed to remove excess storage buffer, blood, or contaminants. Excess liquid can be removed, for example, using a brief centrifugation step, or by other means. The tissue can be frozen as part of the preparation process using any suitable cooling means. For example, the FMC or IAFC can be flash-frozen using liquid nitrogen. Alternatively, the material can be placed in an isopropanol/dry ice bath or can be flash-frozen in other coolants. Commercially available quick-freezing processes can be used. Additionally, the material can be placed in a freezer and allowed to equilibrate to the storage temperature more slowly, rather than being flash-frozen. The tissue can be stored at any desired temperature. For example, −20° C. or −80° C. or other temperatures can be used for storage.

Antimicrobial agents such as antibiotics or anti-fungal agents may be added. The material can be packaged and stored, for example, at room temperature, or for example, at −20° C. or −80° C. prior to use.

In some embodiments, the preparation is present as a dry formulation. A dry formulation can be stored in a smaller volume and may not require the same low temperature storage requirements to keep the formulation from degrading over time. A dry formulation can be stored and reconstituted prior to use. The dry formulation can be prepared, for example, by removing at least a portion of the water in the composition. Water can be removed from the preparation by any suitable means. An exemplary method of removing the water is by use of lyophilization using a commercially available lyophilizer or freeze-dryer. Suitable equipment can be found, for example, through Virtis, Gardiner, N.Y.; FTS Systems, Stone Ridge, N.Y.; and SpeedVac (Savant Instruments Inc., Farmingdale, N.Y.). In certain embodiments, the water content of the dry formulation will be less than about 20%, down to about 10%, down to about 5% or down to about 1% by weight of the formulation. In some embodiments, substantially all of the water is removed. The lyophilized composition can then be stored. The storage temperature can vary from less than about −196° C., −80° C., −50° C., or −20° C. to more than about 23° C. If desired, the composition can be characterized (weight, protein content, etc.) prior to storage.

The lyophilized composition can be reconstituted in a suitable solution or buffer prior to use. Exemplary solutions include but are not limited to PBS, DMEM, and BSS. The pH of the solution can be adjusted as needed. Additional compounds can be added to the composition. Exemplary compounds that can be added to the reconstituted formulation include but are not limited to pH modifiers, buffers, collagen, antibiotics, stabilizers, proteins, and the like (discussed further below).

### B. Enzyme Treatments

In one aspect, the methods involve the treatment of FMC or IAFC with enzymes in order to reduce or remove elastin and blood vessels from the material. The treatment may be simultaneous or sequential and may involve reduction/removal of elastin first followed by reduction/removal of blood vessels or vice versa. Two enzymes specifically contemplated for these methods are pepsin and elastase, which are discussed in detail below.

### 1. Pepsin

Pepsin is an endopeptidase that breaks down proteins into smaller amino acids. It is produced in the chief cells of the stomach lining and is one of the main digestive enzymes in the digestive systems of humans and many other animals, where it helps digest the proteins in food. Pepsin is an aspartic protease, using a catalytic aspartate in its active site. It is one of three principal proteases in the human digestive system, the other two being chymotrypsin and trypsin. Pepsin is most efficient in cleaving peptide bonds between hydrophobic and preferably aromatic amino acids such as phenylalanine, tryptophan, and tyrosine.

Pepsin's proenzyme, pepsinogen, is released by the chief cells in the stomach wall, and upon mixing with the hydrochloric acid of the gastric juice, pepsinogen activates to become pepsin. Pepsin is expressed as a zymogen called pepsinogen whose primary structure has an additional 44 amino acids. In the stomach, chief cells release pepsinogen. This zymogen is activated by hydrochloric acid (HCl), which is released from parietal cells in the stomach lining The hormone gastrin and the vagus nerve trigger the release of both pepsinogen and HCl from the stomach lining when food is ingested. Hydrochloric acid creates an acidic environment, which allows pepsinogen to unfold and cleave itself in an autocatalytic fashion, thereby generating pepsin (the active form). Pepsin cleaves the 44 amino acids from pepsinogen to create more pepsin.

Pepsinogens are mainly grouped in 5 different groups based on their primary structure: pepsinogen A (also called pepsinogen I), pepsinogen B, progastricsin (also called pepsinogen II and pepsinogen C), prochymosin (also called prorennin) and pepsinogen F (also called pregnancy-associated glycoprotein).

Pepsin is most active in acidic environments between 37° C. and 42° C. Accordingly, its primary site of synthesis and activity is in the stomach (pH 1.5 to 2). Pepsin will digest up to 20% of ingested amide bonds by cleaving preferentially at the C-terminal side of aromatic amino acids such as phenylalanine, tryptophan, and tyrosine. Pepsin exhibits preferential cleavage for hydrophobic, preferably aromatic, residues in P1 and P1′ positions. Increased susceptibility to hydrolysis occurs if there is a sulfur-containing amino acid close to the peptide bond, which has an aromatic amino acid. Pepsin cleaves Phe¹Val, Gln⁴His, Glu¹³Ala, Ala¹⁴Leu, Leu¹⁵Tyr, Tyr¹⁶Leu, Gly²³Phe, Phe²⁴ in the insulin B chain. Pepsin exhibits maximal activity at pH 2.0 and is inactive at pH 6.5 and above, however pepsin is not fully denatured or irreversibly inactivated until pH 8.0. Therefore, pepsin in solution of up to pH 8.0 can be reactivated upon re-acidification.

### 2. Elastase

Elastase is an enzyme from the class of proteases (peptidases) that break down proteins. In particular, it is a serine protease and breaks down elastin, an elastic fiber that, together with collagen, determines the mechanical properties of connective tissue. The neutrophil form breaks down the Outer membrane protein A (OmpA) of E. coli and other Gram-negative bacteria. Elastase also has the important immunological role of breaking down Shigella virulence factors. This is accomplished through the cleavage of peptide bonds in the target proteins. The specific peptide bonds cleaved are those on the carboxyl side of small, hydrophobic amino acids such as glycine, alanine, and valine.

### C. Calcification

Once stem cells have invaded, using osteogenic media, the stem cells could be induced to turn into osteoblasts (bone cells). Since in development bone forms from cartilage, scaffolds can be similarly used. These cells would calcify the FMC or IAFC and allow for the production of tissue-engineered bone or if only one part is calcified of cartilage-bone units as present in articular joints.

### D. Other Biological Molecules

In other aspects, the disclosed transplant materials may be treated with additional agent to improve their quality, stability or to support function once transplanted. The bulk FMC or IAFC, with or without cells, can be treated with collagenase, hyaluronidase. TGF-β, endothelial growth factor, platelet derived growth factors, animal serum, platelet lysate, platelet rich plasma, fibriblasts growth factor, stromal derived growth factor, any growth factor, and small molecules to increase cell infiltration, cell differentiation, or in vivo integration. A wide variety of other growth factors, peptides, bioactive small molecules or antimicrobials could be used to recruit or differentiate more cells or keep the FMC or IAFC sterile.

### D. Re-Cellularization

In one embodiment, the transplant material is introduced into a subject without any prior attempt to re-introduce cells and the recipient's own cells will migrate into the transplant in vivo. However, other embodiments the transplant material can be further engineered by reintroducing cells prior to transplantation. This re-cellularized transplant material can be transplanted immediately after reintroducing cells and or can be further cultured to permit the cells to expand, migrate and/or differentiate within the transplant material. Additional factors may be included in the culture system or the transplant material itself to help stimulate expansion, migration and or differentiation. The culturing feature, when employed, may be for as short as a day to about 6 weeks, or even longer.

The cells used to repopulate the transplant materials can be stem cells (such as mesenchymal stem cells), chondrocytes, fibrochondrocytes, cartilage progenitor cells, induced pluripotent stem cells, stem/progenitor cells derived from induced pluripotent stem cells, synovial stem cells, pericytes, pulp/gingival stem cells, adipose derived stem cells, any stem or progenitor cell. These cells may be autologous to said subject, allogenic to said subject, or xenogenic to said subject. The amount of introduced cells prior to culturing (if any) can range from about 1×10³ to about 1×10⁶ cells or more, which can vary based on the ability of the cells to expand.

### E. Buffers

The disclosed transplant materials may be advantageously combined with a buffer solution to maintain a target pH in the processed materials. A buffer solution (more precisely, pH buffer or hydrogen ion buffer) is an aqueous solution consisting of a mixture of a weak acid and its conjugate base, or vice versa. Its pH changes very little when a small amount of strong acid or base is added to it. Buffer solutions are used as a means of keeping pH at a nearly constant value in a wide variety of chemical applications. In nature, there are many systems that use buffering for pH regulation. For example, the bicarbonate buffering system is used to regulate the pH of blood.

The pH of a solution containing a buffering agent can only vary within a narrow range, regardless of what else may be present in the solution. In biological systems this is an essential condition for enzymes to function correctly. For example, in humans a mixture of carbonic acid (H₂CO₃) and bicarbonate (HCO—₃) is present in blood plasma; this constitutes the major mechanism for maintaining the pH of blood between about 7.35 and 7.45. If the pH value of a solution rises or falls too much the effectiveness of an enzyme decreases in a process, known as denaturation, which is usually irreversible. The majority of biological samples that are used in research are kept in a buffer solution, often phosphate buffered saline (PBS) at about pH 7.4.

Some simple buffering agents relevant to physiologic pH include citric acid and KH₂PO₄. By combining substances with pKₐ values differing by only two or less and adjusting the pH, a wide range of buffers can be obtained. Citric acid is a useful component of a buffer mixture because it has three pKₐ values, separated by less than two. The buffer range can be extended by adding other buffering agents. Various McIlvaine's buffer solutions, composed of Na₂HPO₄ and citric acid, have a buffer range of pH 3 to 8. Other commonly used buffers for biological systems include LRS, TRIS, HBSS, GBSS, TAPSO, HEPES, TES, MOPS, PIPES, Cacodylate and MES.

### III. Decellularized Cartilage Compositions

In accordance with the present disclosure, there are provided a decellularized transplant material as described above. The materials have reduced cellular content such that they are substantially acellular, e.g., at least 50% reduction in starting cellular content, and at least 60%, 70%, 80%, 90%, 95% or 99% decellularized. These compositions may be further treated or supplemented with other materials as described herein.

### A. Collagen

In one aspect, the materials may include one or more collagen types. Fibril-forming or network-forming collagens including but not limited to type I, II, III, IV, V, VIII, X, XI, XXIV, or XXVII may be employed as in situ polymerizing gel-forming agents (discussed below). Other collagens may be included in the composition as well.

Collagen is the main structural protein in the extracellular matrix in the various connective tissues in the body. As the main component of connective tissue, it is the most abundant protein in mammals, making up from 25% to 35% of the whole-body protein content. Collagen consists of amino acids bound together to form a triple helix of elongated fibril known as a collagen helix. It is mostly found in fibrous tissues such as tendons, ligaments, and skin.

Over 90% of the collagen in the human body is type I collagen; however, as of 2011, 28 types of collagen have been identified, described, and divided into several groups according to the structure they form. All of the types contain at least one triple helix. The number of types shows collagen's diverse functionality, and any one or more of the following may be included in the disclosed transplant materials:
Fibrillar (Type I, II, III, V, XI, XXIV, XXVII)
Non-fibrillar
FACIT (Fibril Associated Collagens with Interrupted Triple Helices) (Type a, XII,
XIV, XVI, XIX, XX, XXI, XXII)
Network-forming collagens (Type VIII, IV, X)
Multiplexin (Multiple Triple Helix domains with Interruptions) (Type XV, XVIII)
MACIT (Membrane Associated Collagens with Interrupted Triple Helices) (Type XIII, XVII)
Transmembrane associated collagen (XXIII)
Other (Type VI, VII, XXVI, XXVIII)

The five most common types are Type I (skin, tendon, vasculature, organs, bone (main component of the organic part of bone), Type II (cartilage; main collagenous component of cartilage), Type III (reticulate; main component of reticular fibers; commonly found alongside Type I), Type IV (forms basal lamina, the epithelium-secreted layer of the basement membrane) and Type V (cell surfaces, hair, and placenta).

Most medical collagen is derived from young beef cattle (bovine) from certified BSE-free animals. Most manufacturers use donor animals from either “closed herds” or from countries which have never had a reported case of BSE such as Australia, Brazil, and New Zealand.

Collagen scaffolds are used in tissue regeneration, whether in sponges, thin sheets, or gels. Collagen has the correct properties for tissue regeneration such as pore structure, permeability, hydrophilicity, and being stable in vivo. Collagen scaffolds are also ideal for the deposition of cells such as osteoblasts and fibroblasts, and once inserted, growth is able to continue as normal in the tissue.

### B. Glycosaminoglycans

The disclosed transplant materials will also contain glycosaminoglycans, or GAGs. GAGs are long linear polysaccharides consisting of repeating disaccharide (double sugar) units. Except for keratan, the repeating unit consists of an amino sugar, along with a uronic sugar or galactose. Because GAGs are highly polar and attract water, they are used in the body as a lubricant or shock absorber. Mucopolysaccharidoses are a group of metabolic disorders in which abnormal accumulations of glycosaminoglycans occur because of enzyme deficiencies.

Glycosaminoglycans vary greatly in molecular mass, disaccharide construction, and sulfation. This is because GAG synthesis is not template driven like proteins or nucleic acids, but constantly altered by processing enzymes. GAGs are generally classified into four groups based on core disaccharide structures. Heparin/heparan sulfate (HSGAGs) and chondroitin sulfate/dermatan sulfate (CSGAGs) are synthesized in the Golgi apparatus, where protein cores made in the rough endoplasmic reticulum are post-translationally modified with O-linked glycosylation by glycosyltransferases forming proteoglycans. Keratan sulfate may modify core proteins through N-linked glycosylation or O-linked glycosylation of the proteoglycan. The fourth class of GAG, hyaluronic acid is synthesized by integral membrane synthases which immediately secrete the dynamically elongated disaccharide chain.

HSGAG- and CSGAG-modified proteoglycans first begin with a consensus Ser-Gly/Ala-X-Gly motif in the core protein. Construction of a tetrasaccharide linker that consists of -GlcAβ1-3Galβ1-3Galβ1-4Xylβ1-O-(Ser)-, where xylosyltransferase, β4-galactosyl transferase (GalTI), β3-galactosyl transferase (GalT-II), and β3-GlcA transferase (GlcAT-I) transfer the four monosaccharides, begins synthesis of the GAG modified protein. The first modification of the tetrasaccharide linker determines whether the HS GAGs or CSGAGs will be added. Addition of a GlcNAc promotes the addition of HSGAGs while addition of GalNAc to the tetrasaccharide linker promotes CSGAG development. GlcNAcT-I transfers GlcNAc to the tetrasaccahride linker, which is distinct from glycosyltransferase GlcNAcT-II, the enzyme that is utilized to build HSGAGs. EXTL2 and EXTL3, two genes in the EXT tumor suppressor family, have been shown to have GlcNAcT-I activity. Conversely, GalNAc is transferred to the linker by the enzyme GalNAcT to initiate synthesis of CSGAGs, an enzyme which may or may not have distinct activity compared to the GalNAc transferase activity of chondroitin synthase.

With regard to HSGAGs, a multimeric enzyme encoded by EXT1 and EXT2 of the EXT family of genes, transfers both GlcNAc and GlcA for HSGAG chain elongation. While elongating, the HSGAG is dynamically modified, first by N-deacetylase, N-sulfotransferase (NDST1), which is a bifunctional enzyme that cleaves the N-acetyl group from GlcNAc and subsequently sulfates the N-position. Next, C-5 uronyl epimerase coverts d-GlcA to 1-IdoA followed by 2-O sulfation of the uronic acid sugar by 2-O sulfotransferase (Heparan sulfate 2-O-sulfotransferase). Finally, the 6-O and 3-O positions of GlcNAc moities are sulfated by 6-O (Heparan sulfate 6-O-sulfotransferase) and 3-O (3-OST) sulfotransferases.

Chondroitin sulfate and dermatan sulfate, which comprise CSGAGs, are differentiated from each other by the presence of GlcA and IdoA epimers respectively. Similar to the production of HSGAGs, C-5 uronyl epimerase converts d-GlcA to 1-IdoA to synthesize dermatan sulfate. Three sulfation events of the CSGAG chains occur: 4-O and/or 6-O sulfation of GalNAc and 2-O sulfation of uronic acid. Four isoforms of the 4-O GalNAc sulfotransferases (C4ST-1, C4ST-2, C4ST-3, and D4ST-1) and three isoforms of the GalNAc 6-O sulfotransferases (C6ST, C6ST-2, and GalNAc4S-6ST) are responsible for the sulfation of GalNAc.

Unlike HSGAGs and CSGAGs, the third class of GAGs, those belonging to keratan sulfate types, are driven towards biosynthesis through particular protein sequence motifs. For example, in the cornea and cartilage, the keratan sulfate domain of aggrecan consists of a series of tandemly repeated hexapeptides with a consensus sequence of E(E/L)PFPS. Additionally, for three other keratan sulfated proteoglycans, lumican, keratocan, and mimecan (OGN), the consensus sequence NX(T/S) along with protein secondary structure was determined to be involved in N-linked oligosaccharide extension with keratan sulfate. Keratan sulfate elongation begins at the nonreducing ends of three linkage oligosaccharides, which define the three classes of keratan sulfate. Keratan sulfate I (KSI) is N-linked via a high mannose type precursor oligosaccharide. Keratan sulfate II (KSII) and keratan sulfate III (KSIII) are O-linked, with KSII linkages identical to that of mucin core structure, and KSIII linked to a 2-O mannose. Elongation of the keratan sulfate polymer occurs through the glycosyltransferase addition of Gal and GlcNAc. Galactose addition occurs primarily through the β-1,4-galactosyltransferase enzyme (β4Gal-T1) while the enzymes responsible for β-3-Nacetylglucosamine have not been clearly identified. Finally, sulfation of the polymer occurs at the 6-position of both sugar residues. The enzyme KS-Gal6ST (CHST1) transfers sulfate groups to galactose while N-acetylglucosaminyl-6-sulfotransferase (G1cNAc6ST) (CHST2) transfers sulfate groups to terminal GlcNAc in keratan sulfate.

The fourth class of GAG, hyaluronic acid, is not sulfated and is synthesized by three transmembrane synthase proteins HAS1, HAS2, and HAS3. HA, a linear polysaccharide, is composed of repeating disaccharide units of→4)GlcAβ(1→3)GlcNAcβ(1→and has a very high molecular mass, ranging from 10⁵ to 10⁷ Da. Each HAS enzyme is capable of transglycosylation when supplied with UDP-GlcA and UDP-GlcNAc. HAS2 is responsible for very large hyaluronic acid polymers, while smaller sizes of HA are synthesized by HAS1 and HAS3. While each HAS isoform catalyzes the same biosynthetic reaction, each HAS isoform is independently active. HAS isoforms have also been shown to have differing Kₘ values for UDP-GlcA and UDPGlcNAc. It is believed that through differences in enzyme activity and expression, the wide spectrum of biological functions mediated by HA can be regulated, such as its involvement with neural stem cell regulation in the subgranular zone of the brain.

### IV. Methods of Treatment

The decellularized transplant material of the present disclosure can be used for virtually any transplant procedure, such as transplant into the trachea, larynx, rib, ear, nose, hip, epiglottis, intervertebral disc, joint, or meniscus and any other bodily cartilage. It can also be employed in bone to repair a defect serving as a template for calcification. The methods may encompass subjects with congenital or developmental deficits, those suffering from a traumatic injury causing a deficit, or those having undergone resection of a cancerous lesion that results in a deficit.

In a particular embodiment, the disclosed transplant materials are used to treat subglottic stenosis. Subglottic stenosis is defined as the narrowing of the airway immediately below the vocal folds and is primarily caused by the prolonged presence of an endotracheal tube in the airway. Young children are the most severely affected by this disease. Subglottic stenosis in pediatric patients does not only affect the airway, but it also exhibits horrible comorbidities including speech and cognitive underdevelopment, quality of life sacrifices, and in the most severe cases death. Pediatric stenosis occurs in 8.3% of neonatal ICU patients and can be an unbearable burden for patients and families alike.

The treatment for many pediatric subglottic stenosis cases is laryngotracheal reconstruction surgery (LTR). During LTR, a piece of cartilage from the child's rib cage is resected and used to expand the airway at the site of narrowing. The process of resecting rib cartilage is highly invasive for a young child, and this cartilage is not functionally and anatomically similar that of the airway. Furthermore, young children do not have much if any rib cartilage to spare further complicating this procedure. Thus, the transplant materials described herein can be utilized to create a functional alternative to rib cartilage for pediatric airway expansion during LTR.

Laryngotracheal reconstruction surgery are performed using several different techniques. Endoscopic approaches involve inserting instruments through the mouth to reach the airway. Open-airway surgery involves making an incision in the neck. Open-airway surgery may be performed in a single procedure or in multiple procedures. Endoscopic and single-stage open-airway surgeries are generally recommended for mild cases of stenosis, when the airway is not severely narrowed. For more severe cases of stenosis, or where medical conditions exist that may complicate surgery (e.g., heart, lung or neurological conditions), the doctor may recommend a slower, more conservative approach and perform multiple-stage open-airway reconstruction, which involves a series of procedures over the span of a few weeks to several years.

During a single-stage reconstruction, if a tracheostomy tube has been introduced, it must first be removed. The surgeon then widens the airway using precisely shaped pieces of cartilage into the trachea. A temporary tube is inserted through the mouth or nose into the trachea to support the cartilage grafts and will remain in place from a few days to about two weeks. In a double-stage reconstruction, the surgeon performs a first procedure largely as described for a single-stage procedure. However, to provide a framework for the airway to heal, the tracheostomy tube (if present) is left in place or a stent is inserted. During the healing process, which may take about four to six weeks, or even longer, the tube or stent remains in place. In some case, a narrow part of the windpipe may be removed completely and the remaining segments are sewn together. A third type of procedure, called a hybrid or one-and-a-half-stage reconstruction, combines aspects of both single-stage and double-stage reconstruction. In this technique, a single long stent is placed in the existing tracheostomy tube, and a smaller stent is placed through an opening in the trachea to provide a secure, secondary airway during and after the procedure.

Endoscopic laryngotracheal reconstruction is a less invasive procedure. The surgeon inserts surgical instruments and a rod fitted with a light and camera through a rigid viewing tube called a laryngoscope into the mouth and is thus able to to perform the surgery without making any external incisions. In some cases, this approach will permit placement of the grafts for laryngotracheoplasty. In other cases, the surgeon may use lasers, balloons or other methods to relieve the narrowing endoscopically without needing to do a full laryngotracheoplasty. This surgical option may not appropriate for is severely narrowed or scarred airways.

Other uses for the materials disclosed here in include various repair and “patch” embodiments. For example, invertebrate disk herniation and damage to the annulus fibrosus, tympanic membrane perforations and whole tympanic membrane engineering, laryngoesophageal fistulas, tracheostomy patch, or any other injuries which require a biomaterial patch such as skin, burns, wounds, etc., are envisioned.

The invertebrate disk separates adjacent vertebrae and is a joint that allow slight movement in the spine. Each IVD contains 2 regions, the inner region (nucleus pulpous) and the outer region (annulus fibrosus). The nucleus which contains a high concentration of GAGs and is responsible for maintain hydration, osmolarity and osmotic pressure within the disk. The annulus fibrous is made of fibrocartilage with a near identical biochemical composition to meniscal cartilage. Each year there are more than 3 million disc herniations where the anulus fibrous is torn causing leakage in the nucleolus pulpous. Studies have shown that patching annulus fibrous in these injuries with a biomaterial leads to better clinical outcomes. The materials described herein have structural similarity to the anulus fibrous, making it an ideal therapy repair disc herniations. Additionally, it can be used to repair any injury to the annulus fibrosis including engineering a full annulus fibrosus for disk replacement. Further, the process utilized to decellularize and create channels within the meniscal cartilage can be employed to do the same with annulus fibrosis cartilage, which will provide a new potential geometry and fiber orientation option.

The tympanic membrane is a thin fibrous membrane that transduces sound waves into mechanical vibrations. Conductive hearing loss occurs when there is a discontinuity in the mechanical sound transduction chain and is generally caused by overpressure, physical harm, or disease. Of the middle ear anatomy, the tympanic membrane is the most often damaged and has little capacity for self-regeneration. In many cases, surgery is required to place a biomaterial or autologous grafts in place of the tympanic membrane hole. The materials described herein share many mechanical and biochemical similarities to the tympanic membrane and offers a non-invasive option to autologous grafting to repair these types of injuries. Each year there are 150,000 tympanic membrane surgeries in the USA and our technology could represent a better alternative to current treatments. Of particular interest is the ability to choose the fiber alignment, thickness and shape of our technology to match that of native tympanic membrane, something not currently possible.

Laryngoesophageal Fistula (TEF) is a pediatric condition where the esophagus and trachea are abnormally connected. This causes the passage of solid food from the esophagus to the airway. To correct this serious condition, surgeons place some type of sealant or tissue clip to permanently close the connection. However, all of these surgical methods have a high revision rate of up to 4 times before the hole is properly sealed. The disclosed materials offer a more consistent outcome at patching pediatric patients with laryngoesophageal fistulas by offering the proper tissue integration and mechanical properties to the esophagus and trachea. Preliminary rabbit data in laryngotracheal repair indicate that a patch with using the disclosed materials can be sutured in place and fully seal the trachea with no leaks.

The disclosed materials have the benefit of produced in many shapes and sizes with tunable directionality in the channels. This tunability would lend itself to being utilized as an all-purpose biomaterial patch during surgical operations. Much like Biodesign® (decellularized porcine intestinal submucousal) that is used for a wide range of membranous patching applications, the disclosed materials can be utilized in the same way with the advantage of superior mechanical properties and more control on the directionality of cellular invasion or barrier-function.

### V. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Methods

Treatment of FMC material. The cross-sections were devitalized with 4 freeze/thaw cycles. Samples were frozen at −20° C. and thawed to room temperature for one hour each, respectively. The first 2 cycles were conducted dry and the following 2 in hypotonic buffer (10 mM Tris-base, pH 8). Samples were immediately transferred to a vented plastic flask with 0.1% pepsin in 0.5 M acetic acid. The flask was placed in a shaking incubator for 24 hours at 150 rpm and 37° C. After 24 hours, the pepsin solution was removed the samples were washed in 3x PBS for 12 hours total. The PBS was replaced with 0.3 U/mL elastase in 0.2 M Tris-base, pH 8.6 and incubated at 150 rpm and 37° C. for 24 hours. After enzymatic treatment, 3x PBS washes were conducted for 12 hours total. Using a 6 mm biopsy punch, cylinders were punched from the digested cross-sections. These cylinders were punched closest to the posterior of the cross-section to ensure the maximal number of channels. The cylinders were either used immediately or stored at −20° C. for later use.

Preparation of transplant material for cell culture. Sterile digested meniscus pieces were synthesized similar as above with the following modifications: After acid pepsin treatment, meniscus pieces are considered sterile and transferred to a sterile plastic 125 mL Erlenmeyer flask. All reagents following this step were sterile filtered (0.22 micron) and added under a biosafety hood. After the cylinders were punched, then were soaked in 20% fetal bovine serum (FBS) in DMEM for 24 hours. This serves a dual purpose of checking for sterility along with absorbing serum into the meniscus pieces. After 24 hours, the excess FBS was removed via 3x PBS washes. The meniscus cylinders were either used for experimentation immediately or frozen at −20° C. for later use.

Cell introduction. Sterile decellularized, digested meniscus cylinders were placed on top of the membrane insert of a transwell plate. 100 μL of hMSCs in DMEM with 1% PSF (Antibiotic-Antimycotic) at a density ranging from 1×10⁵ to 6×10⁵ were seeded on top of the menisci cylinders. 600 μL of 20% FBS and 1% PSF in DMEM was added to the plate below the inserts to create a gradient of serum ranging from 0-20%. Each day, 50 μL of 1% PSF in DMEM was added on top of the inserts and every 2 days the media on the bottom was exchanged with 600 μL of 20% FBS and 1% PSF in DMEM. After 1 and 2 weeks, the menisci cylinders were removed from the transwell plate and stained with calcein AM. The surface and cross-section of the menisci cylinders were fluorescently imaged using a Keyence BZX microscope and then fixed using 4% paraformaldehyde overnight. H&E staining (2.x) was conducted on the sectioned cylinders.

### Example 2—Results

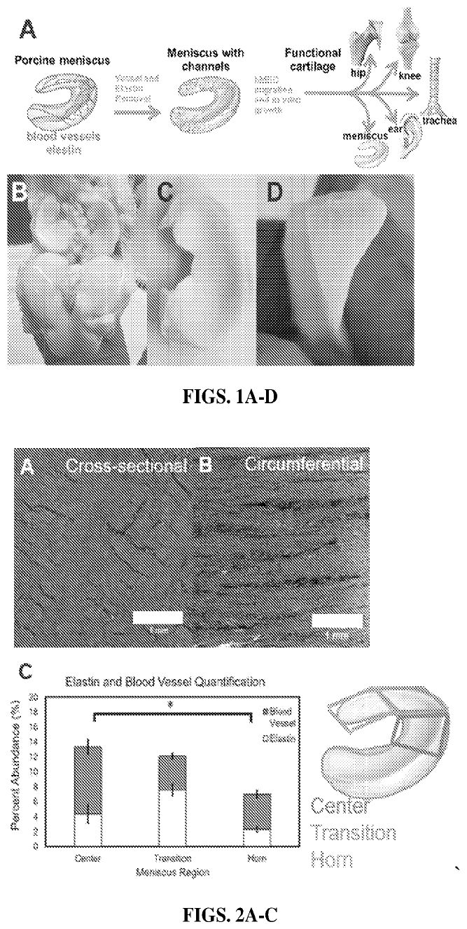

Porcine menisci were extract from whole knees obtained. When sectioned and stained with Verhoeff van-gieson, substantial purple and black shaded regions were observed. These colored areas were indicative of the elastin content surrounding the blood vessels as well as circumferential fibers spanning the tissue (FIGS. 2A & 2B). Using imagej, an area analysis of the purple and black area was conducted and compared to the total area of the tissue. This analysis was used to determine which overall regions of the crescent shaped meniscus had the highest content of blood vessels and elastin. It was shown the center and transition regions had the highest content of these components (FIG. 2C). Using this data, the hypothesis that removing this elastin content and blood vessels could produce channels inside meniscal cartilage for cellular reinvasion and eventual cartilage transplant repair (FIG. 1A).

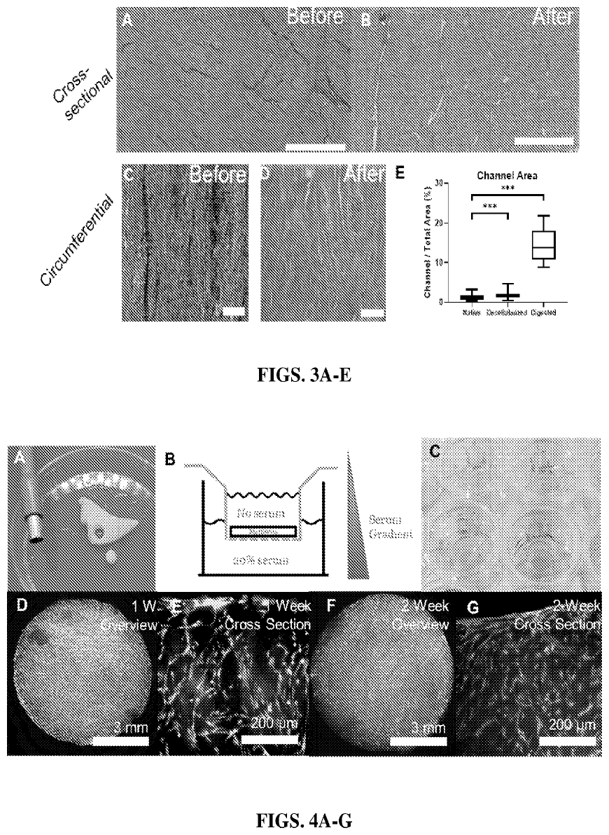

To further characterize removal of the blood vessels and elastin content form the meniscus, histology was conducted before and after enzymatic treatment (pepsin then elastase). Using both Verhoeff van-gieson the presence of channels was confirmed after enzymatic treatment. FIGS. 3A & B clearly show that the purple vessels and elastin are fully removed and resulted in channel formation where the vessels and elastin previously were. Furthermore, in the circumferential orientation the aligned elastin fibers lead to the creation of aligned channels which are ideal for guided cellular reinvasion (FIGS. 3C & D). Blood vessels provide radial access channels. Area image analysis was used to measure the volume of these channels compared to the native and only decellularized tissue. Compared to both the decellularized and native conditions, the enzymatically digest meniscus had a 17.1% and 18.5% more “empty space” respectively. This “empty space” is considered the volume of the channels for reinvasion (FIG. 3E).

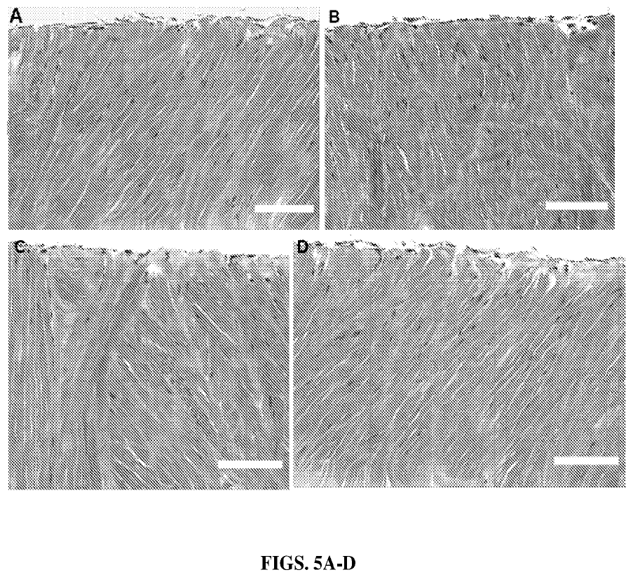

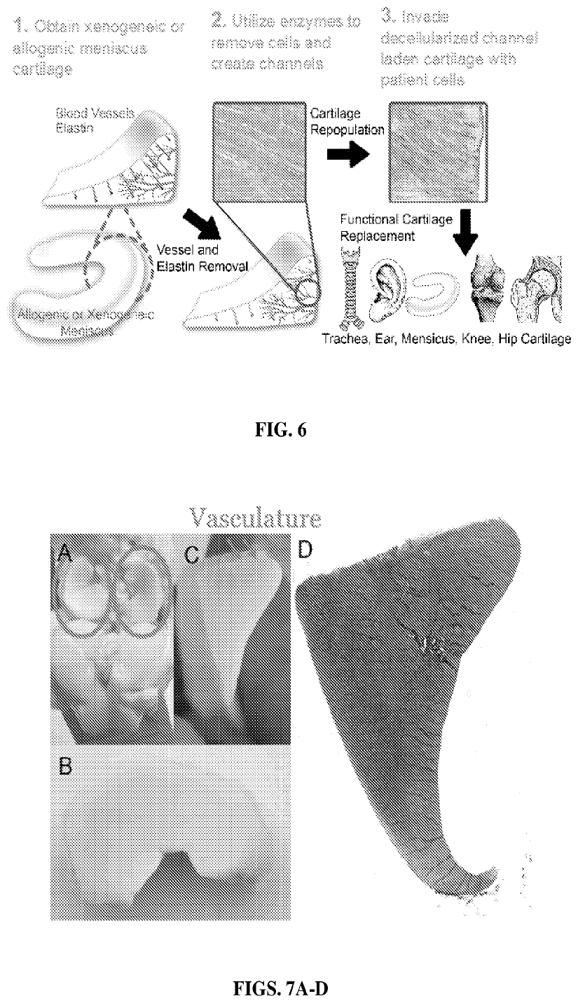

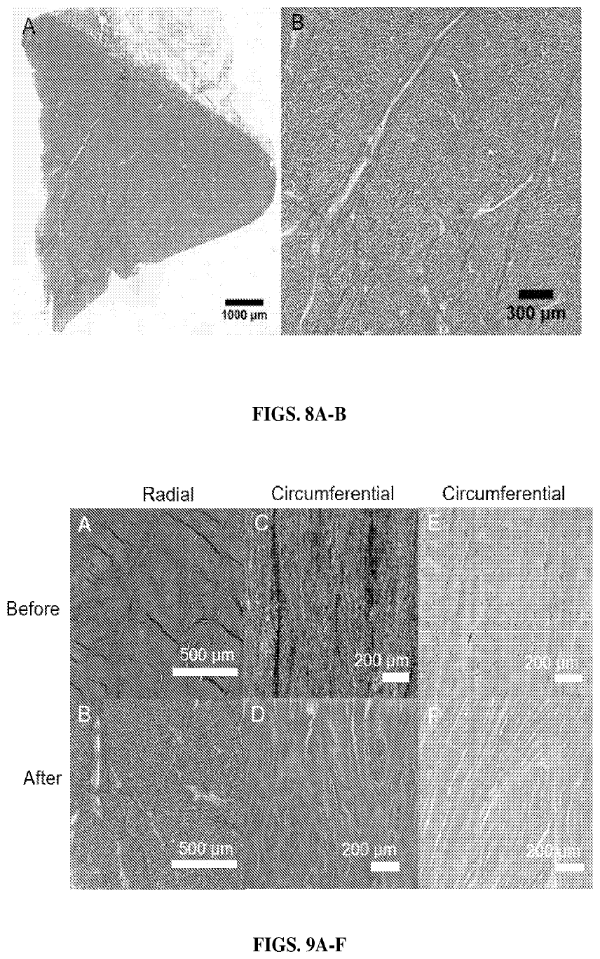

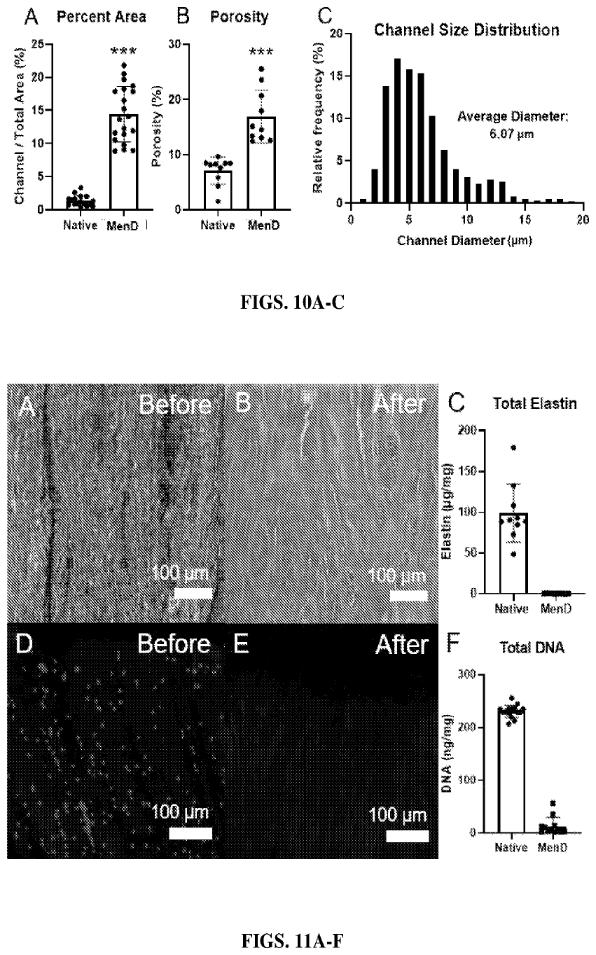

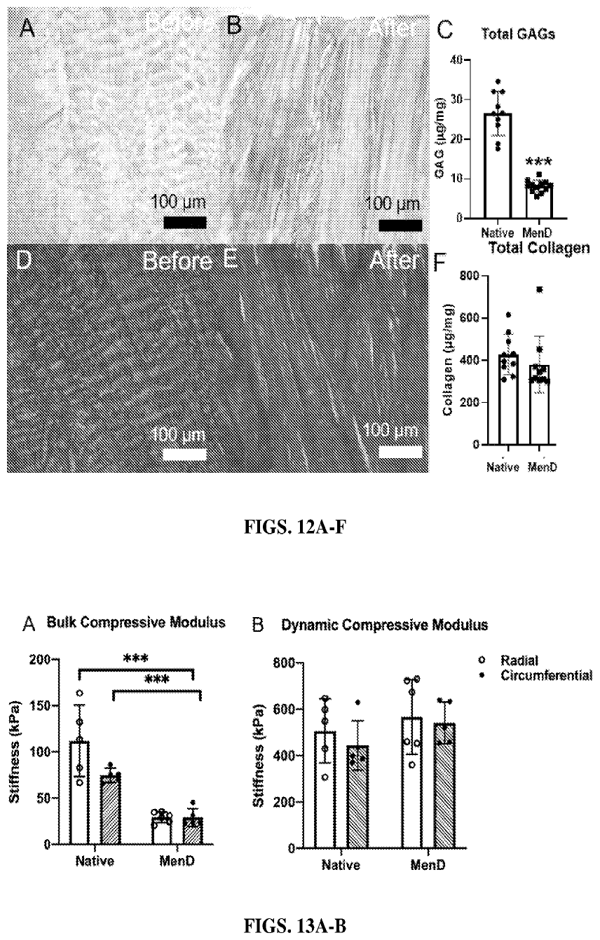

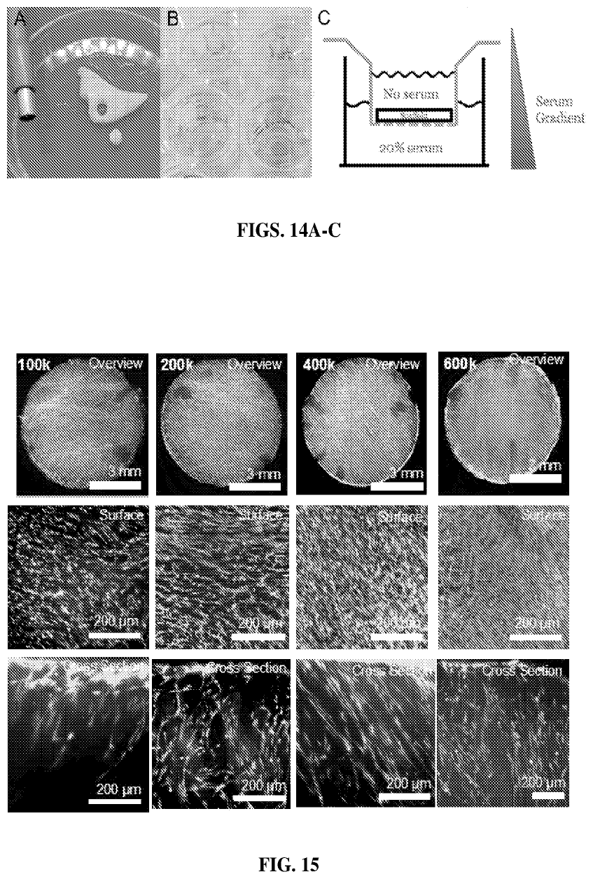

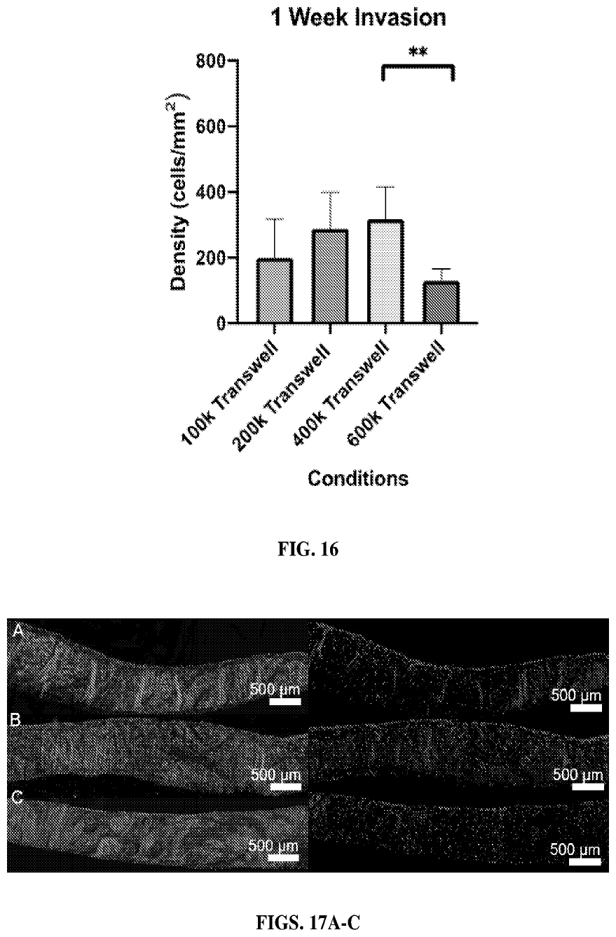

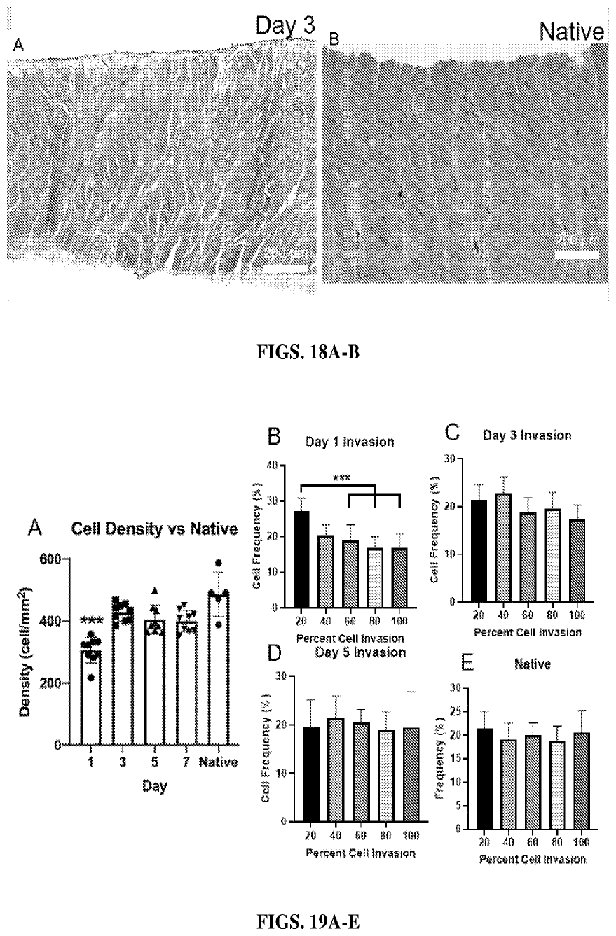

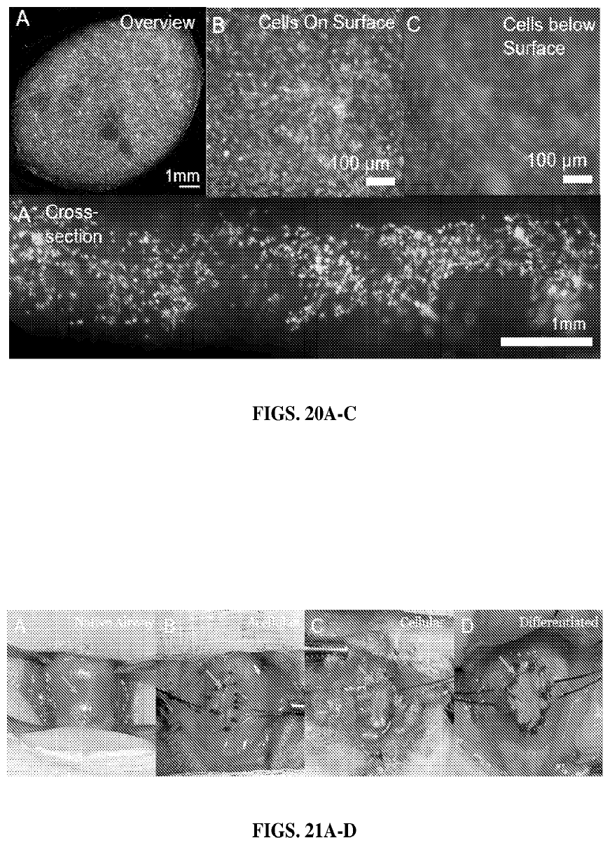

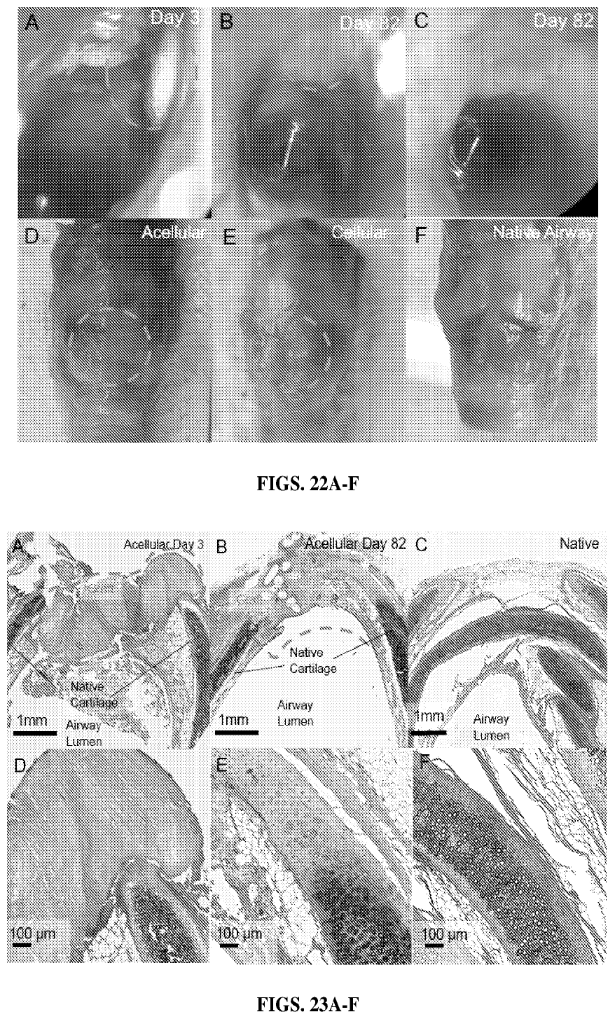

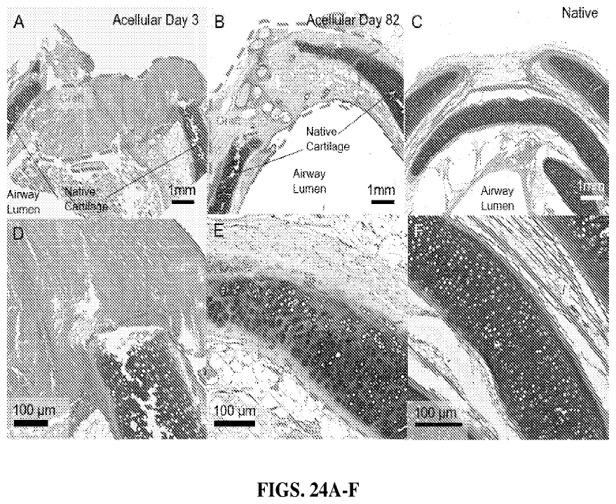

Human bone marrow derived stem cells (hMSCs) were seeded on top of the cross-sectionally punched menisci (FIG. 4A) inside a transwell plate. This transwell plate was utilized to create a serum gradient ranging from 0% to 20% serum to promote cellular chemotaxis through the decellularized channel-laden meniscus (FIGS. 4B & C). After 1 and 2 weeks inside the transwell plate, the cells were alive on top of the meniscus punches as indicated by the live cell stain (calcein AM) (FIGS. 4D & F). Using a scalpel, the stained disks were cut in half and the cross section was imaged. The cross-sections reveal that cells invaded the decellularized channel-laden meniscus and homogenously spread throughout the channels. This result was confirmed by fixing and sectioning the tissue from this experiment. The sections were stained with hematoxylin and eosin and confirmed that cells invaded the entire meniscus (FIGS. 5A-D).

All of the methods disclosed and claimed herein can be made and executed without undue experimentation in light of the present disclosure and the knowledge of one of ordinary skill in this art. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VI. References

The following references, to the extent that they provide exemplary procedural or other details supplementary to those set forth herein, are specifically incorporated herein by reference.

### Lehmann et al., Biofabrication (11) 035012.

### Nurnberger et al., Acta Biomaterials 86:207-222, 2019

1. A method of preparing a decellularized transplant material comprising:
(a) providing fibrous meniscal cartilage (FMC) or intervertebral annulus fibrosis cartilage (IAFC); and
(b) treating said FMC or IAFC with to remove blood vessels and/or elastin to produce said decellularized transplant material.

2. The method of claim 1, wherein step (b) comprises treatment with one or more enzymes such an endopeptidase (e.g., trypsin, chymotrypsin), a cysteine protein (e.g., papain) and in particular with pepsin and/or elastase.

3. The method of claim 1, wherein step (b) comprises treatment with EDTA, EGTA, DCTA, an acid (e.g., acetic acid, hydrochloric acid) or base (e.g., NaOH).

4. The method of claim 1, wherein the FMC or IAFC of step (a) is of human origin or is of non-human origin, such as pig, rabbit, sheep, goat and cow.

5. The method of claim 1, wherein the FMC or IAFC of step (a) is cadaver FMC or IAFC.

6. The method of claim 1, wherein the FMC or IAFC of step (a) is from a living donor.

7. The method of claim 1, wherein the decellularized transplant material is free of at least 50%, 60%, 70%, 75%, 80%, 85%, 90%, 95% or 99% greater of starting elastin content or is 100% devoid of starting elastin content, and/or is free of at least 50%, 60%, 70%, 75%, 80%, 85%, 90%, 95% or 99% greater of starting blood vessel content or is 100% devoid of starting blood vessel content by dry weight.

8. The method of claim 1, wherein the decellularized transplant material is free of at least 50%, 60%, 70%, 75%, 80%, 85%, 90%, 95% or 99% greater of starting cellular content by dry weight.

9. The method of claim 1, wherein the decellularized transplant material is at least 30%, 40%, 50%, 60%, 70%, 75%, 80%, 85%, 90%, 95%, 99% or 100% glycosaminoglycan and collagen by dry weight.

10. The method of claim 1, wherein the FMC or IAFC of step (a) is treated by one or more freeze/thaw cycles.

11. The method of claim 1, wherein step (b) comprises treatment with pepsin followed by treatment with elastin.

12. The method of claim 11, wherein treatment with pepsin is for about 24 hours at 37° C. and/or treatment with elastin is for about 24 hours at 37° C.

13. The method of claim 1, further comprising incubating the decellularized transplant material in serum, such as FBS, a peptide, a protein, a small molecule, a growth factor, or chemically modifying agent.

14. The method of claim 1, further comprising storage about +4 to −80° C.

15. The method of claim 1, further comprising the step of reintroducing cells into said decellularized transplant material following step (b).

16. A decellularized transplant material made according to the method of claim 1.

17. A decellularized transplant material comprising fibrous meniscal cartilage (FMC) or intervertebral annulus fibrosis cartilage (IAFC) that lacks at least 50% of the elastin and blood vessels of normal FMC or or IAFC.

18. The decellularized transplant material of claim 16, wherein the material is of human origin.

19. The decellularized transplant material of claim 16, wherein the material is of non-human origin, such as pig, rabbit, sheep, goat and cow.

20. The decellularized transplant material of claim 16, wherein the material is cadaver FMC or IAFC.

21. The decellularized transplant material of claim 16, wherein the material is from a living donor.

22. The decellularized transplant material of claim 16, wherein the decellularized transplant material is free of at least 50%, 60%, 70%, 75%, 80%, 85%, 90%, 95%, 99% free, or is 100% free, of elastin as compared to untreated material and/or is free of at least 50%, 60%, 70%, 75%, 80%, 85%, 90%, 95%, 99% free, or is 100% free, of blood vessel content as compared to untreated FMC or IAFC by dry weight.

23. The decellularized transplant material of claim 16, wherein the decellularized transplant material is free of at least 50%, 60%, 70%, 75%, 80%, 85%, 90%, 95% or 99% greater of starting FMC or IAFC cellular content by dry weight and/or wherein the decellularized transplant material is at least 30%, 40%, 50%, 60%, 70%, 75%, 80%, 85%, 90%, 95%, 99% or 100% glycosaminoglycan and collagen by dry weight.

24. The decellularized transplant material of claim 16, wherein the decellularized transplant material is re-cellularized with a cell, such as a stem cell (such as mesenchymal stem cells), a progenitor cell, chondrocytes, fibrochondrocytes, cartilage progenitor cells, induced pluripotent stem cells, stem/progenitor cells derived from induced pluripotent stem cells, synovial stem cells, pericytes, pulp/gingival stem cells, or adipose derived stem cells.

25. The decellularized transplant material of claim 16, wherein said material is frozen.

26. A method of transplanting a decellularized transplant material into a living subject comprising:
(a) obtaining a decellularized transplant material according claim 16; and
(b) transplanting said material into said subject.

27. The method of claim 26, further comprising the step of reintroducing cells into said decellularized transplant material prior to step (b).

28. The method of claim 27, wherein the re-cellularized transplant material is transplanted immediately after reintroducing cells and without culture.

29. The method of claim 27, further comprising culturing the re-cellularized transplant material prior to step (b), optionally including the use of one or more factors or conditions that induce cell differentiation/specification.

30. The method of claim 29, wherein culturing is for 1 day to about 3 months.

31. The method of claim 27, wherein the decellularized transplant material is re-cellularized with a stem cell (such as mesenchymal stem cells), a progenitor cell, chondrocytes, fibrochondrocytes, cartilage progenitor cells, induced pluripotent stem cells, stem/progenitor cells derived from induced pluripotent stem cells, synovial stem cells, pericytes, pulp/gingival stem cells, adipose-derived stem cells, fibroblasts, endothelial cells, muscle cells, osteoblasts, osteocytes, osteoclasts, macrophages, monocytes, or cells of the immune system.

32. The method of claim 27, wherein said re-introduced cells are autologous to said subject.

33. The method of claim 32, wherein the autologous cells are genetically engineered or modified, such as iPSCs.

34. The method of claim 27, wherein said re-introduced cells are allogenic to said subject.

35. The method of claim 27, wherein about 1×10³ to about 1×10⁸ cells are reintroduced.

36. The method of claim 26, wherein said decellularized transplant material is autologous to said subject.

37. The method of claim 26, wherein said decellularized transplant material is allogenic to said subject.

38. The method of claim 26, wherein said decellularized transplant material is xenogenic to said subject.

39. The method of claim 26, wherein said subject is a non-human animal.

40. The method of claim 26, wherein said subject is a human.

41. The method of claim 40, wherein said human subject is a human pediatric subject.

42. The method of claim 26, wherein said decellularized transplant material is transplanted into trachea, larynx, rib, ear (e.g., tympanic membrane), nose, hip, knee, temporomandibular joint, epiglottis, intervertebral disc, a joint, or meniscus.

43. The method of claim 26, wherein said decellularized transplant material is transplanted into a bone defect or soft issue defect.

44. The method of claim 26, wherein said subject has suffered a traumatic injury.

45. The method of claim 26, wherein said subject has undergone surgery, such as resection of a cancerous lesion or tracheostomy.

46. The method of claim 26, wherein the decellularized transplant material is transplanted as a treatment for disk herniation, tympanic membrane damage, laryngoesophageal fistula, cleft palate, osteoarthritis, spine fusion, joint overuse, a birth defect (e.g., CHARGE syndrome), or microtia, or as an alveolar bone graft.
